(19) United States
(12) Patent Application Publication
(10) Pub. No.: US 2009/0182337 A1
(43) Pub. Date: Jul. 16, 2009

(54) Antimicrobial Coatings

(76) Inventors: Joshua B. Stopek, Yalesville, CT (US); Joseph Hotter, Middletown, CT (US); Steve Tsai, Stamford, CT (US)

Correspondence Address: Tyco Healthcare Group LP, 60 MIDDLETOWN AVENUE, NORTH HAVEN, CT, 06473, US

(21) Appl. No.: 12/300,573

(22) PCT Filed: May 14, 2007
(86) PCT No.: PCT/US07/11645

§ 371 Date: Nov. 12, 2008

Related U.S. Application Data
(60) Provisional application No. 60/800,387, filed on May 15, 2006.

(51) Int. Cl. A01N 43/08; A01P 1/00; A61L 17/00; A61B 17/064; A61B 17/03; A61F 2/82; A61B 17/58; A61F 13/00

(52) U.S. Cl. 606/70; 514/473; 606/231; 606/219; 606/151; 623/1.46; 606/76; 602/48

(57) ABSTRACT

An antimicrobial coating is provided for use on textiles, medical devices, packaging materials, and the like. The antimicrobial coating includes a halogenated furanone.

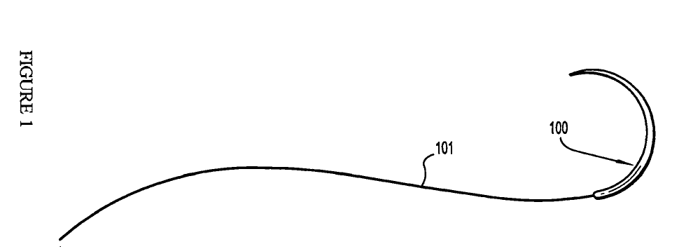

### CROSS-REFERENCE TO RELATED APPLICATIONS

This application claims the benefit of U.S. Provisional Patent Application No. 60/800,387, filed May 15, 2006, the entire disclosure of which is incorporated by reference herein.

### TECHNICAL FIELD

The present disclosure relates to antimicrobial coatings suitable for use on textiles, medical devices, packaging materials, and the like.

### BACKGROUND OF RELATED ART

The use of antimicrobial agents on textiles is known. See, e.g., U.S. Patent Application Publication No. 2003/0204916. The use of antimicrobial agents on medical devices such as sutures and/or packages containing said sutures has also been previously disclosed. However, some medical devices may not provide effective levels of antimicrobial activity for a sufficient period of time. Moreover, as is apparent from U.S. Patent Application Publication Nos. 2004/0068293 and 2004/0068294, antimicrobial agents on medical devices can be undesirably transferred to their packages, requiring the use of higher levels of antimicrobial agents in order to obtain the desired antimicrobial effect upon implantation of the suture or other medical device in vivo.

Accordingly, there is a need for medical devices, packaging materials and textiles that can retain enhanced antimicrobial efficacy. There is also a need for an easy and inexpensive method of applying antimicrobial agents to a medical device, packaging material or textile that provides protection against microorganisms for extended periods of time, with minimal loss of the antimicrobial agents from the article surface and/or minimal transference of the antimicrobial agent to packaging materials, etc. In this way, lower amounts of antimicrobial agents may be utilized to achieve the desired antimicrobial effect.

### SUMMARY

The present disclosure provides articles possessing a coating including a film-forming polymer in combination with a halogenated furanone. Articles which may possess such a coating include medical devices, packaging materials and textiles. In embodiments, the coating may also include one or more fatty acid components such as a fatty acid, a fatty acid salt, and/or a salt of a fatty acid ester.

In other embodiments, the present disclosure provides antimicrobial compositions including halogenated furanones, a glycolide/caprolactone copolymer, and a fatty acid component such as a fatty acid, a fatty acid salt, and/or a salt of a fatty acid ester.

Sutures having antimicrobial properties are also provided. In embodiments, such a suture has an elongate structure and a coating material disposed on said elongate structure, the coating including a film-forming polymer and a halogenated furanone. In embodiments, the coating may also include a fatty acid component such as a fatty acid, a fatty acid salt, and/or a salt of a fatty acid ester.

Methods for closing wounds are also provided. In embodiments, a method of closing a wound includes providing a suture possessing a coating including a film-forming polymer in combination with a halogenated furanone on at least a portion of the suture, attaching said suture to a needle to produce a needled suture, and passing said needled suture through tissue to create wound closure.

### BRIEF DESCRIPTIONS OF THE DRAWINGS

The FIGURE is a depiction of a needled suture in accordance with the present disclosure.

### DETAILED DESCRIPTION

The present disclosure provides coatings suitable for textiles, medical devices, packaging materials, and the like. The coatings include polymers in combination with halogenated furanones. In embodiments, fatty acids, salts of fatty acids and/or salts of fatty acid esters may be added to the coatings.

Any polymer suitable for use as a coating may be utilized in accordance with the present disclosure. Polymers may be bioabsorbable or nonabsorbable. In embodiments, a bioabsorbable film-forming polymer may be utilized in a coating of the present disclosure. Film-forming polymers which may be utilized in the coating are within the purview of those skilled in the art and include those containing linkages derived from monomers including, for example, glycolide, lactide, glycolic acid, lactic acid, caprolactone, trimethylene carbonate, dioxanones, dioxepanones, and the like, and homopolymers, copolymers and combinations thereof.

In embodiments, the film-forming polymer may include a caprolactone containing copolymer as described in U.S. Pat. No. 5,716,376, the entire disclosure of which is incorporated by reference herein. Such a caprolactone containing copolymer can be obtained by polymerizing a major amount of epsilon-caprolactone and a minor amount of at least one other copolymerizable monomer or mixture of such monomers in the presence of a hydroxyl-functional initiator, such as a polyhydric alcohol initiator.

Monomers which can be copolymerized with epsilon-caprolactone include alkylene carbonates such as trimethylene carbonate, tetramethylene carbonate, dimethyl trimethylene carbonate; dioxanones; dioxepanones; absorbable cyclic amides; absorbable cyclic ether-esters derived from crown ethers; hydroxyacids capable of esterification, including alpha hydroxy acids (such as glycolic acid and lactic acid) and beta hydroxyacids (such as beta hydroxybutyric acid and gamma hydroxyvaleric acid); polyalkyl ethers (such as polyethylene glycol), and combinations thereof. In embodiments, glycolide can be utilized as the comonomer with epsilon-caprolactone in the film-forming polymer.

Suitable polyhydric alcohol initiators which may be utilized in preparing the film-forming polymer include glycerol, trimethylolpropane, 1,2,4-butanetriol, 1,2,6-hexanetriol, triethanolamine, triisopropanolamine, erythritol, threitol, pentaerythritol, ribitol, arabinitol, xylitol, N,N,N′,N′-tetrakis(2-hydroxyethyl)ethylenediamine, N,N,N′,N′-tetrakis(2-hydroxypropyl)ethylenediamine, dipentaerythritol, allitol, dulcitol, glucitol, altritol, iditol, sorbitol, mannitol, inositol, and the like; with mannitol being utilized in some embodiments.

The polyhydric alcohol initiator can be employed in small amounts, in embodiments from about 0.01 to about 5 weight percent of the total monomer mixture, in other embodiments from about 0.1 to about 3 weight percent of the total monomer mixture.

Where utilized, the film-forming copolymer can contain from about 70 to about 98 weight percent epsilon-caprolactone derived units, in embodiments from about 80 to about 95 weight percent epsilon-caprolactone derived units, the balance of the copolymer being derived from the other copolymerizable monomer(s), such as glycolide.

Coatings of the present disclosure also include halogenated furanones. Halogenated furanones are known as inhibitors of quorum sensing. Quorum sensing, also known as bacterial signaling, is recognized as a general mechanism for gene regulation in many bacteria, and it allows bacteria to perform in unison such activities as bioluminescence, swarming, biofilm formation, production of proteolytic enzymes, synthesis of antibiotics, development of genetic competence, plasmid conjugal transfer, and spoliation. Quorum sensing is a universal regulatory mechanism used by both Gram-positive bacteria such as Staphylococcus aureus, Streptococcus pneumoniae, Salmonella enteritidis, Staphylococcus epidermidis, Bacillus subtilis, and the like, and Gram-negative bacteria such as Pseudomonas aeruginosa, Escherichia coli, Aeromonas hydrophila, and the like.

Thus, a quorum sensing inhibitor, such as the halogenated furanones described herein, may act as an antimicrobial agent by inhibiting microbial development and proliferation. In embodiments, a quorum sensing inhibitor may inhibit swarming motility and biofilm formation, both of which frequently underlie the pathophysiology of infectious diseases. The inhibition of swarming and biofilm formation may thus reduce bacterial burden and hence prevent infection and disease progression.

Halogenated furanones may also block quorum sensing and inhibit the growth of bacteria at amounts that are non-toxic to mammalian cells. Given their mechanism of action, halogenated furanones' antipathogenic properties may be effective against a broad spectrum of infectious agents and may be able to reduce and/or prevent colonization of both gram positive and gram negative bacteria, including those noted above.

In addition, unlike antibiotics and antiseptic compounds which kill microbes and carry the risk of inducing antimicrobial resistance, halogenated furanones do not exert such evolutionary pressures. Thus, antimicrobial resistance to an article coated with a composition of the present disclosure including a halogenated furanone is not a concern.

Suitable halogenated furanones for use in coatings of the present disclosure include, for example, compounds of the following formula:

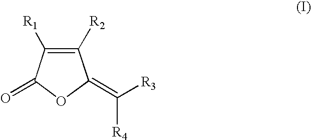

wherein R₂, R₃ and R₄ are independently or all H or halogen;

“═” represents a double bond; and

R₁ is a moiety such as H, halogen, formyl, carboxyl, cyano, ester, amide, alkyl, alkoxy, oxoalkyl, alkenyl, alkynyl, aryl or arylalkyl, which moiety may optionally be substituted with one or more substituents; and/or interrupted by one or more hetero atoms; and/or straight chain, branched chain, hydrophobic, hydrophilic or fluorophilic,

provided that at least one of R₁, R₂, R₃ and R₄ is a halogen. Any halogen may be utilized; in embodiments, at least one of R₁, R₂, R₃ and R₄ is bromine.

As used herein, a substituted furanone or substituted moiety includes one possessing a group such as alkyl, cycloalkyl, alkenyl, alkynyl, halo, haloalkyl, haloalkynyl, hydroxy, alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenacyl, alkynylacyl, acylamino, diacylamino, acyloxy, alkylsulfonyloxy, heterocyclyl, heterocycloxy, heterocyclamino, haloheterocyclyl, alkylsulfenyl, carboalkoxy, alkylthio, acylthio, and/or phosphorus-containing groups such as phosphono and phosphinyl.

As used herein, “alkyl”, used either alone or in compound words such as “haloalkyl” or “alkylthio”, includes straight chain or branched C₁₋₆ alkyl groups. Examples include methyl, ethyl, propyl, isopropyl and the like.

As used herein, “alkoxy” includes straight chain or branched alkoxy, in embodiments C₁₋₁₀ alkoxy such as methoxy, ethoxy, n-propoxy, isopropoxy and butoxy isomers.

As used herein, “alkenyl” includes groups formed from straight chain, branched or mono- or polycyclic alkenes including ethylenically mono- or poly-unsaturated alkyl or cycloalkyl groups as previously defined, in embodiments C₂₋₁₀ alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, or 1,3,5,7-cyclooctatetraenyl.

As used herein, “halogen” and/or “halogenated” includes fluorine, chlorine, bromine and/or iodine.

As used herein, “heteroatoms” include O, N and/or S.

As used herein, “acyl” used either alone or in compound words such as “acyloxy”, “acylthio”, “acylamino” or diacylamino” includes carbamoyl, aliphatic acyl groups and acyl groups containing a heterocyclic ring which may be referred to as heterocyclic acyl, in embodiments C₁₋₁₀ acyl. Examples of acyl include carbamoyl; straight chain or branched alkanoyl, such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl; alkoxycarbonyl, such as methoxycarbonyl, ethoxycarbonyl, t-butoxycarbonyl, t-pentyloxycarbonyl or heptyloxycarbonyl; cycloalkylcarbonyl such as cyclopopylcarbonyl cyclobutylcarbonyl, cyclopentylcarbonyl or cyclohexylcarbonyl; alkylsulfonyl, such as methylsulfonyl or ethylsulfonyl; alkoxysulfonyl, such as methoxysulfonyl or ethoxysulfonyl; heterocyclylcarbonyl; heterocyclylalkanoyl, such as pyrrolidinylacetyl, pyrrolidinylpropanoyl, pyrrolidinylbutanoyl, pyrrolidinylpentanoyl, pyrrolidinylhexanoyl or thiazolidinylacetyl; heterocyclylalkenoyl, such as heterocyclylpropenoyl, heterocyclylbutenoyl, heterocyclylpentenoyl or heterocyclylhexenoyl; or heterocyclylglyoxyloyl, such as, thiazolidinylglyoxyloyl or pyrrolidinylglyoxyloyl.

As used herein, “fluorophilic” includes the highly attractive interactions certain groups, such as highly fluorinated alkyl groups of C₄-C₁₀ chain length, have for perfluoroalkanes and perfluoroalkane polymers.

In embodiments, specific halogenated furanones which may be utilized in coatings of the present disclosure include, for example, the following brominated furanones:

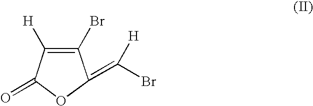

also known as 4-bromo-5-(bromomethylene)-2(5H)-furanone;

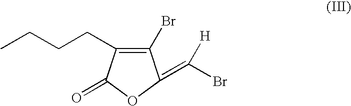

also known as (5Z)-4-bromo-5-(bromomethylene)-3-butyl-2(5H)-furanone;

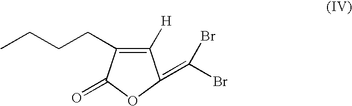

also known as 5-(dibromomethylene)-3-butyl-2(5H)-furanone;

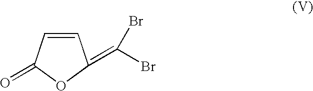

also known as 5-(dibromomethylene)-2(5H)-furanone;

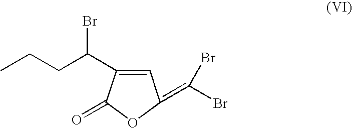

also known as 3-(1′-bromobutyl)-5-(dibromomethylene)-2(5H)-furanone;

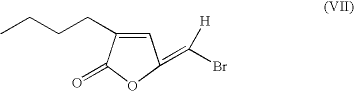

also known as (5Z)-5-(bromomethylene)-3-butyl-2(5H)-furanone;

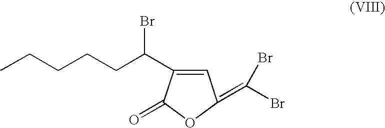

also known as 3-(1′-bromohexyl)-5-(dibromomethylene)-2(5H)-furanone; and

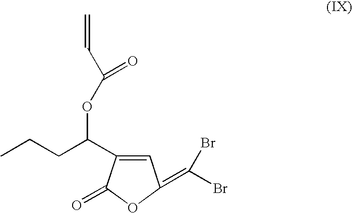

also known as 1-(5-(dibromomethylene)-2-oxo-2,5-dihydrofuran-3-yl) butyl acrylate.

In embodiments, combinations of the foregoing halogenated furanones, optionally in combination with additional halogenated furanones encompassed by formula (I) above, may also be utilized in a coating of the present disclosure.

Any suitable amount of halogenated furanone may be utilized in a coating of the present disclosure. As noted above, due to their excellent antibacterial activity, halogenated furanones may be utilized in low level dosages which are capable of imparting anti-microbial properties to the article to which the coating is applied. In embodiments, the amount of halogenated furanone present in a coating of the present disclosure may be from about 5 parts per million (ppm) to about 1000 ppm, in embodiments from about 20 ppm to about 800 ppm, in other embodiments from about 100 ppm to about 600 ppm. The exact amount of the halogenated furanone in the antimicrobial coating will depend upon a number of factors, such as the particular furanone used, the composition of the article being contacted, and the choice of polymer utilized in the coating material.

In some embodiments, the coating compositions of the present disclosure may also contain a fatty acid component such as a fatty acid, a fatty acid salt, or a salt of a fatty acid ester. Suitable fatty acids may be saturated or unsaturated, and include higher fatty acids having more than about 12 carbon atoms. Suitable saturated fatty acids include, for example, stearic acid, palmitic acid, myristic acid and lauric acid. Suitable unsaturated fatty acids include oleic acid, linoleic acid, and linolenic acid. In addition, an ester of fatty acids, such as sorbitan tristearate or hydrogenated castor oil, may be used.

Suitable fatty acid salts include the polyvalent metal ion salts of C₆ and higher fatty acids, in embodiments those having from about 12 to about 22 carbon atoms, and mixtures thereof. Fatty acid salts including the calcium, magnesium, barium, aluminum, and zinc salts of stearic, palmitic and oleic acids may be useful in some embodiments of the present disclosure. Some useful salts include commercial “food grade” calcium stearate which contains a mixture of about one-third C₁₆ and two-thirds C₁₈ fatty acids, with small amounts of the C₁₄ and C₂₂ fatty acids.

Suitable salts of fatty acid esters which may be included in the bioactive coatings of the present disclosure include calcium, magnesium, aluminum, barium, or zinc stearoyl lactylate; calcium, magnesium, aluminum, barium, or zinc palmityl lactylate; and/or calcium, magnesium, aluminum, barium, or zinc olelyl lactylate. In embodiments; calcium stearoyl-2-lactylate (such as the calcium stearoyl-2-lactylate commercially available under the tradename VERV from American Ingredients Co., Kansas City, Mo.) may be utilized. Other fatty acid ester salts which may be utilized include lithium stearoyl lactylate, potassium stearoyl lactylate, rubidium stearoyl lactylate, cesium stearoyl lactylate, francium stearoyl lactylate, sodium palmityl lactylate, lithium palmityl lactylate, potassium palmityl lactylate, rubidium palmityl lactylate, cesium palmityl lactylate, francium palmityl lactylate, sodium olelyl lactylate, lithium olelyl lactylate, potassium olelyl lactylate, rubidium olelyl lactylate, cesium olelyl lactylate, and francium olelyl lactylate.

Where utilized, the amount of fatty acid component can be from about 5 percent to about 60 percent by weight of the total coating composition. In embodiments, the fatty acid component may be present in an amount from about 15 percent to about 55 percent by weight of the total coating composition.

In one embodiment, the film-forming polymer, such as the caprolactone/glycolide copolymer described above, can be present in an amount from about 45 to about 60 weight percent of the coating and the fatty acid component, such as a fatty acid salt or a salt of a fatty acid ester, can be present in an amount from about 40 to about 55 weight percent of the coating. In embodiments, the film-forming polymer, such as the caprolactone/glycolide copolymer described above, can be present in an amount from about 50 to about 55 weight percent of the coating and the fatty acid component can be present in an amount from about 45 to about 50 weight percent of the coating.

Preparing the antimicrobial coating of the present disclosure may be a relatively simple procedure. For example, the desired amount of a film-forming polymer and a halogenated furanone, optionally in combination with a fatty acid component, may be placed into a container and mixed thoroughly to combine the ingredients. In embodiments, the halogenated furanone may be added to the film forming polymer with no additional additives so that the halogenated furanone is present in the resulting coating in amounts from about 5 ppm to about 1000 ppm, in embodiments from about 20 ppm to about 800 ppm, in other embodiments from about 100 ppm to about 600 ppm.

In one embodiment, the antimicrobial coating can be a suspension formed by mixing a glycolide and caprolactone copolymer with calcium stearoyl lactate at a weight ratio of approximately 52/48, adding methylene chloride, ethanol, and hexane while mixing, and then adding at least one halogenated furanone so that the halogenated furanone may be present in a resulting coating in an amount from about 5 ppm to about 1000 ppm, in embodiments from about 20 ppm to about 800 ppm, in other embodiments from about 100 ppm to about 600 ppm.

In other embodiments, the coating of the present disclosure can be applied as a solution and the solvent evaporated to leave the coating components, in embodiments, a film-forming polymer and a halogenated furanone. Suitable solvents which may be utilized in forming the solution include any solvent or combination of solvents suitable for the chosen coating composition. To be suitable, the solvent should (1) be miscible with the coating components, and (2) not appreciably affect the integrity of any material used to form the article being coated, such as a suture. Some examples of suitable solvents include alcohols, ketones, ethers, aldehydes, acetonitrile, acetic acid, methylene chloride, chloroform and water. In embodiments, methylene chloride may be used as a solvent.

Preparing a coating solution of the present disclosure is also a relatively simple procedure and can be accomplished by blending, mixing, and the like. In one embodiment, where a film-forming polymer, halogenated furanone and methylene chloride are utilized to form the coating solution, the desired amount of film-forming polymer and halogenated furanone may be placed into a container, followed by the addition of the desired amount of methylene chloride. The two ingredients may then be mixed thoroughly to combine the ingredients. In embodiments, a fatty acid component as described above, including a calcium stearoyl lactate, may be included in the coating solution.

Any known technique may be employed for applying the coating, for example as a solution or suspension, to an article. Suitable techniques include dipping, spraying, wiping and brushing. The article wetted with the coating solution or suspension may be subsequently passed through or held in a drying oven for a time and at a temperature sufficient to vaporize and drive off the solvent.

Articles coated with a coating of the present disclosure may be formed from any material in need of improved resistance to bacteria. Such articles include, but are not limited to, textiles, packaging materials, medical devices, and the like.

Textiles which may be coated with coatings of the present disclosure include those made of natural fibers, synthetic fibers, blends of natural fibers, blends of synthetic fibers, and blends of natural fibers with synthetic fibers. Suitable materials utilized to form textiles include polyesters, polyamides, polyolefins, halogenated polymers, polyester/polyethers, polyurethanes, homopolymers thereof, copolymers thereof, and combinations thereof. Specific examples of suitable materials include polyethylene, polypropylene, polybutylene, polyvinyl chloride, polyethylene terephthalate, nylon 6, and nylon 6,6.

Packaging materials which may be coated with coatings of the present disclosure include packaging for products such as medical devices, pharmaceuticals, textiles, consumer goods, foods, and the like. Packaging materials may be formed of any suitable material within the purview of those skilled in the art.

Medical devices which may be coated with a coating of the present disclosure include, but are not limited to, sutures, staples, meshes, patches, slings, stents, grafts, clips, pins, screws, rivets, tacks, bone plates, drug delivery devices, wound dressings, woven devices, non-woven devices, braided devices, adhesion barriers, tissue scaffolds, and other implants.

Medical devices can be formed from any material that has suitable physical properties for the intended use of the medical device. Medical devices can thus be formed of absorbable materials, nonabsorbable materials, and combinations thereof. Suitable absorbable materials which may be utilized to form the medical device include trimethylene carbonate, caprolactone, dioxanone, glycolic acid, lactic acid, glycolide, lactide, homopolymers thereof, copolymers thereof, and combinations thereof. Suitable non-absorbable materials which may be utilized to form the medical device include polyolefins, such as polyethylene, polypropylene, copolymers of polyethylene and polypropylene, and blends of polyethylene and polypropylene.

In one embodiment, a medical device treated in accordance with the present disclosure may be a suture. Sutures in accordance with the present disclosure may be monofilament or multifilament and may be made of any conventional material, including both bioabsorbable and non-bioabsorbable materials, such as surgical gut, silk, cotton, polyolefins such as polypropylene, polyamides, polyglycolic acids, polyesters such as polyethylene terephthalate and glycolide-lactide copolymers, etc.

In one embodiment, the suture may be made of a polyolefin. Suitable polyolefins include polyethylene, polypropylene, copolymers of polyethylene and polypropylene, and blends of polyethylene and polypropylene. In some embodiments, polypropylene can be utilized to form the suture. The polypropylene can be isotactic polypropylene or a mixture of isotactic and syndiotactic or atactic polypropylene.

In other embodiments, the suture may be made from synthetic absorbable polymers such as those made from glycolide, lactide, caprolactone, alkylene carbonates (i.e., trimethylene carbonate, tetramethylene carbonate, etc.), dioxanones, and copolymers and combinations thereof. One combination which may be utilized includes glycolide and lactide based polyesters, including copolymers of glycolide and lactide.

As noted above, the suture can be monofilament or multifilament. Where the suture is a monofilament, methods for producing such sutures are within the purview of those skilled in the art. Such methods include forming a suture material, such as a polyolefin resin, and extruding, drawing and annealing the resin to form the monofilament.

Where the sutures are made of multiple filaments, the suture can be made using any technique within the purview of one skilled in the art such as, for example, braiding, weaving or knitting. The filaments may also be combined to produce a non-woven suture. The filaments themselves may be drawn, oriented, crinkled, twisted, comingled or air entangled to form yarns as part of the suture forming process.

In embodiments a multifilament suture of the present disclosure can be produced by braiding. The braiding can be done by any method within the purview of those skilled in the art. For example, braid constructions for sutures and other medical devices are described in U.S. Pat. Nos. 5,019,093, 5,059,213, 5,133,738, 5,181,923, 5,226,912, 5,261,886, 5,306,289, 5,318,575, 5,370,031, 5,383,387, 5,662,682, 5,667,528, and 6,203,564, the entire disclosures of each of which are incorporated by reference herein.

Once the suture is constructed, it can be sterilized by any means within the purview of those skilled in the art.

In some cases a tubular braid, or sheath, can be constructed about a core structure which is fed through the center of a braider. Known tubular braided sutures, including those possessing cores, are disclosed, e.g., in U.S. Pat. Nos. 3,187,752, 3,565,077, 4,014,973, 4,043,344, and 4,047,533.

Textiles, including individual fibers and fabrics made of multiple fibers, may be formed and/or coated in a similar manner.

A suture coated with a coating of the present disclosure will possess antimicrobial properties. In embodiments, a suture of the present disclosure may possess an elongate structure and be formed from at least one polymeric filament, in embodiments, multiple filaments. The filaments may be formed from a polymeric material that is absorbable under physiological conditions, and a coating of the present disclosure may be placed thereon.

In embodiments, a suture in accordance with the present disclosure may be attached to any surgical needle within the purview of those skilled in the art to produce a needled suture. Such a needled suture is depicted in the FIGURE, with suture 101 attached to needle 100. Wounds may be sutured by passing a needled suture through tissue to create wound closure. The needle may then be removed from the suture and the suture tied. The suture may remain in the tissue and help prevent contamination and infection of said tissue by virtue of its antimicrobial properties, thereby promoting wound healing and minimizing infection. The suture coating also advantageously enhances the surgeon's ability to pass the suture through tissue, and increases the ease and security with which he/she can tie the suture.

Medical devices and packaging materials in accordance with this disclosure can be sterilized in accordance with techniques within the purview of those skilled in the art.

Coatings of the present disclosure, including halogenated furanones described herein, remain attached to the surface of the article during the processing, handling, and storage of the article. This minimizes the loss or transfer of the halogenated furanones from an article to any packaging, from any packaging to any article, the environment, etc.

If desired, the coating composition of the present disclosure can optionally contain additional components, e.g., dyes, antimicrobial agents, growth factors, anti-inflammatory agents, and the like. The term “antimicrobial agent” as used in the present disclosure includes antibiotics, antiseptics, disinfectants and combinations thereof. In embodiments, the antimicrobial agent may be an antiseptic, such as triclosan.

Classes of antibiotics that can be used in the coating include tetracyclines like minocycline; rifamycins like rifampin; macrolides like erythromycin; penicillins like nafcillin; cephalosporins like cefazolin; beta-lactam antibiotics like imipenem and aztreonam; aminoglycosides like gentamicin and TOBRAMYCIN®; chloramphenicol; sulfonamides like sulfamethoxazole; glycopeptides like vancomycin; quinolones like ciprofloxacin; fusidic acid; trimethoprim; metronidazole; clindamycin; mupirocin; polyenes like amphotericin B; azoles like fluconazole; and beta-lactam inhibitors like sulbactam.

In other embodiments, silver salts, including silver salts of ionic furanones, may be added for their antimicrobial properties.

Examples of antiseptics and disinfectants which may be utilized in the coating include hexachlorophene; cationic biguanides like chlorhexidine and cyclohexidine; iodine and iodophores like povidone-iodine; halo-substituted phenolic compounds like PCMX (i.e., p-chloro-m-xylenol) and triclosan (i.e., 2,4,4′-trichloro-2′hydroxy-diphenylether); furan medical preparations like nitrofurantoin and nitrofurazone; methenamine; aldehydes like glutaraldehyde and formaldehyde; and alcohols. In some embodiments, at least one of the antimicrobial agents may be an antiseptic, such as triclosan.

The antimicrobial coating of the present disclosure may contain various optional ingredients, such as stabilizing agents, thickeners, colors, etc. The optional ingredients may represent up to about 10% of the total weight of the antimicrobial coating.

As low amounts of halogenated furanones are required in coatings of the present disclosure, existing formulations and manufacturing processes need only minimal modifications to produce the coatings described herein. This ease of formulation and production may reduce both the time and cost necessary to prepare coatings of the present disclosure, compared with adding other antimicrobial agents to existing coating materials.

While the above description contains many specifics, these specifics should not be construed as limitations on the scope of the disclosure herein but merely as exemplifications of particularly useful embodiments thereof. Those skilled in the art will envision many other possibilities within the scope and spirit of the disclosure as defined by the claims appended hereto.

What is claimed is:

1. An article possessing a coating comprising a film-forming polymer in combination with a halogenated furanone, wherein the article is selected from the group consisting of medical devices, packaging materials and textiles.

2. The article of claim 1, wherein the film-forming polymer contains linkages derived from one or more monomers selected from the group consisting of glycolide, lactide, caprolactone, trimethylene carbonate, dioxanones, dioxepanones, homopolymers thereof, copolymers thereof, and combinations thereof.

3. The article of claim 1, wherein the film-forming polymer comprises a glycolide/caprolactone copolymer.

4. The article of claim 1 wherein the halogenated furanone comprises a compound of formula: 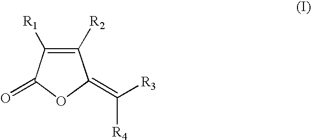
wherein R₂, R₃ and R₄ are independently or all H or halogen;
“═” represents a double bond; and
R₁ is a moiety selected from the group consisting of H, halogen, formyl, carboxyl, cyano, ester, amide, alkyl, alkoxy, oxoalkyl, alkenyl, alkynyl, aryl or arylalkyl, which moiety may optionally be substituted by one or more substituents or interrupted by one or more hetero atoms,
wherein at least one of R₁, R₂, R₃ and R₄ is a halogen.

5. The article of claim 1, wherein the halogenated furanone is selected from the group consisting of 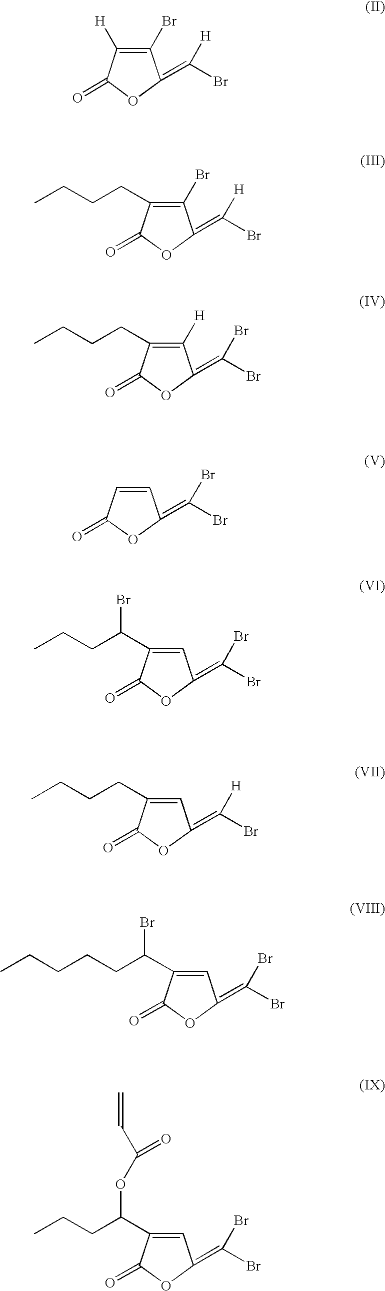
and combinations thereof.

6. The article of claim 1, wherein the halogenated furanone is present in the coating in an amount from about 5 parts per million to about 1000 parts per million.

7. The article of claim 1, wherein the coating further comprises one or more fatty acid components selected from the group consisting of fatty acids, fatty acid salts and salts of fatty acid esters.

8. The article of claim 7, wherein the fatty acid component comprises a salt of a fatty acid ester selected from the group consisting of calcium stearoyl lactylate, magnesium stearoyl lactylate, aluminum stearoyl lactylate, barium stearoyl lactylate, zinc stearoyl lactylate calcium palmityl lactylate, magnesium palmityl lactylate, aluminum palmityl lactylate, barium palmityl lactylate, or zinc palmityl lactylate, calcium olelyl lactylate, magnesium olelyl lactylate, aluminum olelyl lactylate, barium olelyl lactylate, and zinc olelyl lactylate.

9. The article of claim 1 wherein the textile is selected from the group consisting of natural fibers, synthetic fibers, blends of natural fibers, blends of synthetic fibers, and blends of natural fibers with synthetic fibers.

10. The article of claim 9 wherein the textile is selected from the group consisting of polyesters, polyamides, polyolefins, halogenated polymers, polyester/polyethers, polyurethanes, homopolymers thereof, copolymers thereof, and combinations thereof.

11. The article of claim 1, wherein the packaging material is for a product selected from the group consisting of medical devices, pharmaceuticals, textiles, consumer goods and foods.

12. The article of claim 1 wherein the medical device is selected from the group consisting of sutures, staples, meshes, patches, slings, stents, grafts, clips, pins, screws, rivets, tacks, bone plates, drug delivery devices, wound dressings, woven devices, non-woven devices, braided devices, adhesion barriers, and tissue scaffolds.

13. The article of claim 1 wherein the coating further comprises at least one antimicrobial agent selected from the group consisting of antibiotics, antiseptics, disinfectants and combinations thereof.

14. An antimicrobial composition comprising:
a halogenated furanone;
a glycolide/caprolactone copolymer; and
a fatty acid component selected from the group consisting of fatty acids, fatty acid salts and salts of fatty acid esters.

15. A suture comprising an elongate structure and a coating material disposed on at least a portion of said elongate structure, said coating comprising:
a film-forming polymer; and
a halogenated furanone.

16. The suture of claim 15, wherein the film-forming polymer contains linkages derived from one or more monomers selected from the group consisting of glycolide, lactide, caprolactone, trimethylene carbonate, dioxanones, dioxepanones, homopolymers thereof, copolymers thereof, and combinations thereof.

17. The suture of claim 15, wherein the halogenated furanone comprises a compound of formula: 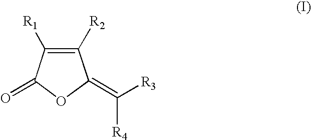
wherein R₂, R₃ and R₄ are independently or all H or halogen;
“═” represents a double bond; and
R₁ is a moiety selected from the group consisting of H, halogen, formyl, carboxyl, cyano, ester, amide, alkyl, alkoxy, oxoalkyl, alkenyl, alkynyl, aryl or arylalkyl, which moiety may optionally be substituted by one or more substituents or interrupted by one or more hetero atoms,
wherein at least one of R₁, R₂, R₃ and R₄ is a halogen.

18. The suture of claim 15, wherein the coating further comprises a fatty acid component selected from the group consisting of fatty acids, fatty acid salts and salts of fatty acid esters, and wherein the film-forming polymer comprises a glycolide/caprolactone copolymer.

19. The suture of claim 18, wherein the salt of a fatty acid ester is selected from the group consisting of calcium stearoyl lactylate, magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, zinc palmityl lactylate, calcium olelyl lactylate, magnesium olelyl lactylate, aluminum olelyl lactylate, barium olelyl lactylate, and zinc olelyl lactylate.

20. A method of closing a wound comprising:
attaching a suture of claim 15 to a needle to produce a needled suture; and
passing said needled suture through tissue to create wound closure.
